(19) United States
(12) Patent Application Publication
(10) Pub. No.: US 2010/0055729 A1
(43) Pub. Date: Mar. 4, 2010

(54) SENSOR DEVICE

(76) Inventor: Dong June Ahn, Seoul (KR)

Correspondence Address: EDWARDS ANGELL PALMER & DODGE LLP, P.O. BOX 55874, BOSTON, MA, 02205, US

(21) Appl. No.: 12/199,736

(22) Filed: Aug. 27, 2008

(51) Int. Cl. C12Q 1/02; C12M 1/34; G01N 33/48; G01N 33/68; B01J 19/00; G01N 21/01; G01N 27/00; G01N 27/26

(52) U.S. Cl. 435/29; 435/287.1; 436/94; 436/86; 436/89; 422/68.1; 422/82.01; 422/82.05; 204/400

(57) ABSTRACT

Sensor devices and compositions can be used for detecting the presence of a target substance in a medium. The sensor devices and compositions can be configured to include at least one nanosensor through a high concentration of nanosensors that interact with the target substance to provide a detectable signal as an indication of such an interaction. The interaction is evidence that the medium includes the target substance. Various types of target substances can be detected with the sensor device, including inorganic molecules, organic molecules, atoms, ions, polypeptides, polynucleotides, cells, derivatives thereof, and combinations thereof.

### BACKGROUND

The detection of one or more specific entities in a composition, as well as in a particular environment, has been a goal in various technical areas. Medical diagnostics have employed the detection of specific entities, such as insulin, cancerous cell receptors, and the like, in order to determine the presence or progression of a disease state. Military and homeland security organizations have employed the detection of specific chemical agents in order to screen for agents that may be hazardous (e.g., poisons, explosives, and the like) or may be precursors for hazardous materials (e.g., reagents for making drugs, poisons, explosives, and the like). While a wide range of detection technologies exists, as evidenced by the medical and security devices currently employed, many of the detection equipment and procedures lack in sensitivity and/or efficient usability.

Recently, many different types of sensor strategies have been devised and employed on a nanoscale. These nanosensors are nano-sized and/or nano-structured materials that have the ability to generate a signal when stimulated by a specific type or genus of stimuli. Often, the nanosensors are configured to be chemosensors that sense when a specific type or genus of a substance interacts with a recognition substrate associated with the nanosensor. Typically, the nanosensors are configured to sense electronic, optical, magnetic, and/or electrochemical signal changes upon recognition of a target substance. The signal output data of the nanosensors can be measured for detection of one or more specific substances.

In many cases high sensitivity of the nanosensor is required due to the dilute concentration of the target in a specific medium. For example, certain biological agents may be present in dilute concentrations that are difficult to detect without high sensitivity. In another example, explosives or explosive residues or reagents may be present on a sample in very small amounts, where detection requires high sensitivity. Without a sufficient level of sensitivity, the ability to detect a target substance may not be achievable. Additionally, background noise contamination can also necessitate the importance of sensitivity.

### SUMMARY

Generally, a sensor device can be used for detecting a target substance in a medium. The sensor device can include a porous polymeric container, and at least one nanosensor included and retained within the porous polymeric container. The nanosensor can be configured to interact with a target substance so as to provide a signal that can be detected. The target substance can be any type of substance. Non-limiting examples of a suitable target substance can include an organic molecule, inorganic molecule, atom, ion, nucleotide, polynucleotide, amino acid, polypeptide, protein, receptor, antibody, antibody fragment, cell, cell surface component, ligand, combinations thereof, or the like. When the target substance is a target polynucleotide, the nanosensor can include a probe polynucleotide configured to hybridize with the target polynucleotide. When the target substance is a target polypeptide, the nanosensor can include a target recognition moiety configured to interact with the target polypeptide. When the target substance is a target cell, the nanosensor can include a target recognition moiety configured to interact with a cell surface component of the target cell. Non-limiting examples of cell surface components include a protein, epitope, receptor, cell membrane component, lipid, combinations thereof, or the like.

In one embodiment, the sensor device can be configured in accordance with at least one of the following: the nanosensor is retained within an internal chamber of the porous polymeric container; the nanosensor is retained within a pore of the porous polymeric container; the nanosensor is coupled to the porous polymeric container; the pores of the porous polymeric container fluidly couple an external environment with an internal chamber of the polymeric container; the pores of the porous polymeric container are dimensioned to be larger than a target substance and smaller than a nanosensor such that the target substance can pass through the pores so as to interact with the nanosensor; or the porous polymeric container can include a biocompatible and biostable polymer.

In one embodiment, a sensor device can include a barcode quantum material configured for multiplexing. The barcode quantum material can be included within the porous polymeric container. Optionally, the nanosensor can include a barcode quantum material that is configured to interact with the target substance.

In one embodiment, the nanosensors can be configured in accordance with at least one of the following: the signal provided by the target substance interacting with the nanosensor is selected from the group consisting of an electronic signal, optical signal, magnetic signal, electrochemical signal, and combinations thereof; the interaction between the target substance and a nanosensor induces a detectable change in the signal; the nanosensor includes an oligonucleotide probe configured to interact with the target substance being a nucleotide sequence; the nanosensor includes a target substance recognition moiety; the nanosensor includes a polypeptide receptor configured to interact with the target substance; the nanosensor includes a bio-barcode configured to release barcode oligonucleotides upon interaction of a nanosensor of the plurality with the target substance; the nanosensor includes a quantum dot; the nanosensor includes a barcode quantum material; the nanosensor includes a nanotube coupled to a target substance recognition moiety such that interaction of the target substance and the recognition moiety changes the detected signal of the nanotube; the nanosensor includes a nano-gap capacitor configured to change the detected signal upon interaction of the target substance and a nanosensor of the plurality; or the nanosensor includes a nano-cantilever coupled to a target substance recognition moiety such that interaction of the target substance and the recognition moiety changes the detected signal of the nano-cantilever.

In one embodiment, a sensor device that detects polynucleotides can include a porous polymeric container, and at least one nanosensor that detects polynucleotides included and retained within the porous polymeric container. The nanosensor can include a probe polynucleotide configured to hybridize with a target polynucleotide. Also, the probe polynucleotide of the nanosensor can have a high degree of specificity for the target polynucleotide, the high degree of specificity being characterized by at least 90% complementarity.

In one embodiment, a sensor device that detects polypeptides can include a porous polymeric container, and at least one nanosensor that detects polypeptides included and retained within the porous polymeric container. The nanosensor can include a target recognition moiety configured to interact with a target polypeptide. The target recognition moiety can be, but is not limited to, one of a polypeptide, protein, receptor, antibody, antibody fragment, ligand, combinations thereof, or the like.

In one embodiment, a sensor device that detects cells can include a porous polymeric container, and at least one nanosensor that detects cells included and retained within the porous polymeric container. The nanosensor can include a target recognition moiety configured to interact with a cell surface component of a target cell. Non-limiting examples of a cell surface component include a protein, epitope, receptor, cell membrane component, lipid, combinations thereof, or the like.

In one embodiment, a sensor device can be included in a sensor system that also includes a monitor that detects a signal provided by the interaction of the nanosensor and the target substance. The monitor can be any monitor that can detects at least one signal selected from an electronic signal, optical signal, magnetic signal, electrochemical signal, and combinations thereof. Also, the monitor can be any monitor that can detect a change in the signal that occurs from the interaction between the target substance and a nanosensor.

In one embodiment, a sensor device as described herein can be used to detect the presence of a target substance. Generally, the sensor device can include a polymeric container having a nanosensor retained therein. The sensor device can be placed in any medium that may or may not have the target substance. In some instances, the sensor device is used to confirm the presence of a target substance in a medium. In other instances, the sensor device is used to confirm the absence of a target substance in a medium. In any event, a signal provided by a target substance interacting with the nanosensor of the sensor device can be detected so as to indicate the presence of the target substance in the medium. While the medium can be any type of medium, non-limiting examples include water, air, biological sample, hydrocarbon, combinations, and the like. In some detection methods, the target substance can be tagged with a marker that interacts with the nanosensor of the sensor device so as to provide a detectable signal. Also, the monitoring or measurement of the signal can be used in determining an amount or concentration of the target substance in the medium. In some instances, a probe signal is directed into the medium in order to induce the nanosensor to provide the signal.

In one embodiment, a sensor device can be manufactured by placing a nanosensor within a container. Such manufacturing can include providing a nanosensor and a porous polymeric container, and combining the nanosensor and the porous polymeric container such that the nanosensor is retained within the porous polymeric container.

In one embodiment, the method of manufacturing can include configuring the nanosensor to interact with a target substance so as to provide a signal that can be detected. Non-limiting examples of target substances that the nanosensor can be configured to interact with include an organic molecule, inorganic molecule, atom, ion, nucleotide, polynucleotide, amino acid, polypeptide, protein, receptor, antibody, antibody fragment, cell, cell surface component, ligand, combinations thereof, or the like. Accordingly, the method of manufacturing can include configuring the nanosensor to include a probe polynucleotide that hybridizes with a target polynucleotide. Additionally, the method of manufacturing can include configuring the nanosensor to include a target recognition moiety that interacts with a target polypeptide. Also, the method of manufacturing can include configuring the nanosensor to include a target recognition moiety that interacts with a cell surface component of a target cell. Non-limiting examples of cell surface components can include a protein, epitope, receptor, cell membrane component, lipid, or combinations thereof.

In one embodiment, the method of manufacture can include at least one of the following: preparing the porous polymeric container to have an internal chamber and including the nanosensor in the internal chamber; configuring pores of the porous polymeric container to have dimensions smaller than dimensions of the nanosensor such that the nanosensor is retained within the porous polymeric container; coupling the nanosensor to the porous polymeric container; configuring pores of the porous polymeric container to be larger than a target substance and smaller than the nanosensor such that the target substance can pass through the pores so as to interact with the nanosensor; including a barcode quantum material configured for multiplexing within the porous polymeric container; or preparing the porous polymeric container from a biocompatible and biostable polymer.

In one embodiment, the polymeric and nanosensor can be combined by at least one of the following: mixing the nanosensor into a polymeric material that is prepared into the polymeric container; extruding a composition having the nanosensors and a polymeric composition; spraying a polymeric composition onto the nanosensor; spraying a nanosensor composition onto a polymer composition; aggregating nanosensors together with a removable aggregator, dipping the aggregated nanosensors into a polymeric composition, and removing the aggregator so as to disaggregate the nanosensors; lyophilizing a solution having the nanosensors and a polymeric composition; inkjet printing a polymeric composition onto the nanosensors; inkjet printing a nanosensor composition onto a polymeric member; depositing a polymeric composition onto the nanosensors; or polymerizing a polymeric precursor in the presence of the nanosensors.

These and other embodiments and features of the sensor device will become more fully apparent from the following description and appended claims, or may be learned by the practice of the sensor device as set forth hereinafter.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

To further clarify the above and other advantages and features of the sensor device and compositions, an illustrative description of the sensor device will be rendered by reference to the appended drawings. It is appreciated that these drawings depict only illustrated embodiments of the sensor device and are therefore not to be considered limiting of its scope.

FIGS. 1A-1E are schematic representations of an illustrative embodiment of a sensor device.

FIG. 2A-2C are schematic representations of an illustrative embodiment of a sensor device having nanosensors that interact with a target substance to provide a detectable signal.

FIGS. 3A-3B are a side view and cutaway side view, respectively, of an illustrative embodiment of a porous sensor device.

FIG. 4 is a cutaway side view of an illustrative embodiment of a sensor device having nanosensors that interact with a target polynucleotide to provide a detectable signal.

FIG. 5 is a cutaway side view of an illustrative embodiment of a sensor device having nanosensors that interact with a target polypeptide to provide a detectable signal.

FIG. 6 is a cutaway side view of an illustrative embodiment of a sensor device having nanosensors that interact with a target cell to provide a detectable signal.

FIG. 7 is a schematic representation of an illustrative embodiment of a nanosensor.

FIG. 8 is a schematic representation of an illustrative embodiment of a nanosensor.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Generally, sensor devices and compositions as described herein can be used for detecting the presence of a target substance in a medium. The sensor devices and compositions can be configured to include a high concentration of nanosensors that interact with the target substance to provide a detectable signal as an indication of such an interaction. The high concentration of nanosensors can be achieved by placing a number of nanosensors into a single container that holds and retains the nanosensors. The container can have various configurations that provide for the retention of the nanosensors when the container is placed within a medium, and that also allows for the target substance to enter the container so as to interact with the nanosensors. The high concentration of nanosensors allows for increased sensitivity in a detection system and method of detecting the presence of the target substance. Also, the high concentration of nanosensors can allow for the detection of the target substance even when present in extremely dilute quantities.

The high concentration of nanosensors is optional because the sensor device can include only one nanosensor. However, the sensor device can include about or at least about 10 nanosensors, at least about 50 nanosensors, at least about 100 nanosensors, at least about 1,000 nanosensors, at least about 10,000 nanosensors, at least about 100,000 nanosensors, at least about 1 million nanosensors, at least about 10 million nanosensors, at least 100 million nanosensors, or at least 1 billion nanosensors. Also, the sensor device can include from about 1 nanosensor about 10 nanosensors, from about 10 nanosensors to about 50 nanosensors, from about 50 nanosensors to about 100 nanosensors, from about 100 nanosensors to about 1,000 nanosensors, from about 1,000 nanosensors to about 10,000 nanosensors, from about 10,000 nanosensors to about 100,000 nanosensors, from about 100,000 nanosensors to about 1 million nanosensors, from about 1 million nanosensors to about 10 million nanosensors, from about 10 million nanosensors to about 100 million nanosensors, or from 100 million nanosensors to about 1 billion nanosensors.

### I. Sensor Device

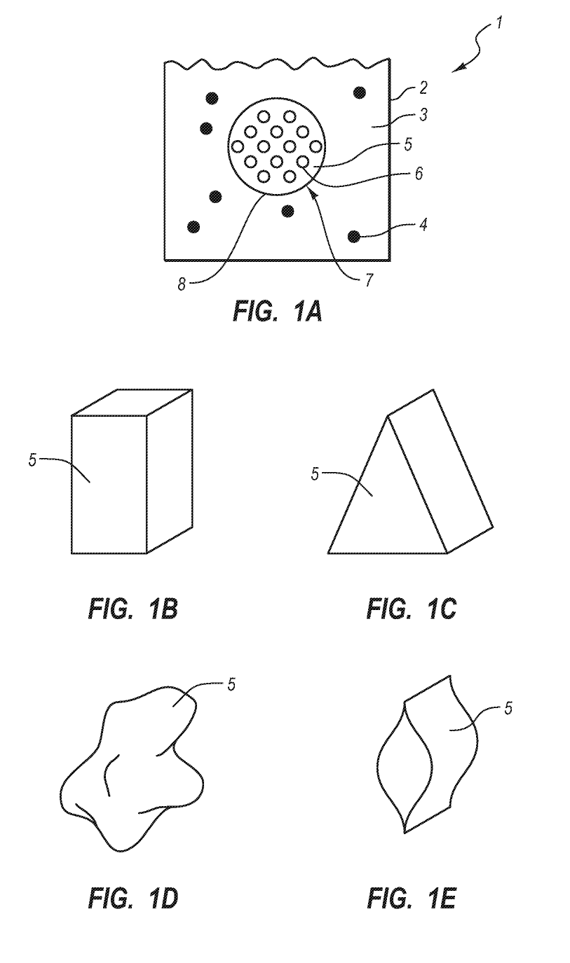

FIG. 1A illustrates an embodiment of a sensor device 1 having a container 5 that is configured as described herein to include a nanosensor (not shown) that can detect the presence of a target substance 4 in an environmental medium 3. As shown, the environmental medium 3 can be a sample within a sample vessel 2 that may or may not include the target substance 4. The container 5 can include a body 7 having a shape defined by an external surface 8 with a plurality of conduits 6 or pores extending into the container 5. The shape shown for the container 5 is substantially spherical; however, other shapes are possible. FIG. 1B-1E show the container 5 having the shape of a block 5, triangle 5, amorphous shape 5, and pouch 5; however, any possible shape can be used. The individual conduits 6 can be configured with any shape and size sufficient for receiving the target substance 4 into the container 5. Also, the container 5 can be made from a polymeric material.

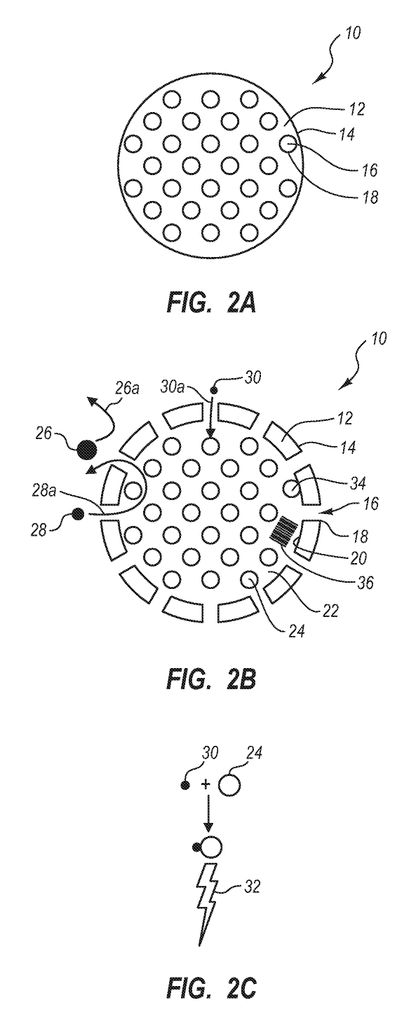

A general configuration of a sensor device 10 is shown in FIGS. 2A-2C. FIG. 2A, which is a side view, shows the sensor device 10 can include a container 12 having a polymeric body that defines an external surface 14 that is perforated with pores 16. Each pore is defined by at least one pore wall 18. The pores 16 can also be holes, conduits, entrances, exits, or the like.

As shown in FIG. 2B, which is a cutaway side view of FIG. 2A, the pores 16 can extend from an external environment into an internal chamber 22 that is defined by at least one internal surface 20. The chamber 22 is dimensioned so as to include at least one nanosensor 24 retained therein, and shown to include a plurality of nanosensors 24.

The pores 16 can be configured to form at least one conduit that opens to the outside of the container 12 and extends to a location within the container 12 that retains the nanosensor 24. The pore can be any type of pore 16 or pore system, or other similar configuration that allows for the medium outside of the container 12 to flow into the chamber 22 so as to interact with the nanosensor 24 retained therein. The pores 16 and container 12 can be configured such that the nanosensor 24 is retained in the chamber 22 in a manner so that the nanosensor 24 is capable of interacting with the target substance 30 to provide a detectable signal. Accordingly, when the container 12 includes a high concentration of nanosensors 24, or a large number of nanosensors, the sensing ability and sensitivity is increased. For example, without limitation, the increase in sensitivity can be doubled, tripled, quadrupled, a logarithmic increase, or any other suitable and detectable increase in sensitivity.

The pores 16 can be shaped, sized, and/or dimensioned to perform size exclusion selection on the substances that can enter into the chamber 22. Also, the material of the container 12 and/or pore walls 18 can be selected to perform affinity and/or exclusion selection for substances, so as to function similarly to material in affinity or exclusion chromatography by having a charge, hydrophobicity, hydrophilicity, and the like. That is, the pores 16 can be configured to restrict substances of a certain size from entering into the pores 16 and/or into the internal chamber 22. Accordingly, the pores 16 allow substances smaller than a certain size to enter into the pores 16 and/or into the internal chamber 22. The size of the pores 16 can be configured to be similar to the target substance 30, which can restrict access to the nanosensors and increase the accuracy of detection. Non-limiting examples of pores sizes include being about or less than about 0.1 nm, less than about 1 nm, less than about 10 nm, less than about 100 nm, less than about 1 um, less than about 10 um, and less than about 100 um. Additional non-limiting examples of pores sizes include being about 0.01 nm to about 0.1 nm, about 0.1 nm to about 1 nm, about 1 nm to about 10 nm, about 10 nm to about 100 nm, about 100 nm to about 1 um, about 1 um to about 10 um, and about 19 um to about 100 um.

In another measurement, non-limiting examples of pore sizes can include a diameter or other dimension that is less than about 99% of the diameter or dimension of the nanosensor, less than or about 90% of the diameter or dimension of the nanosensor, less than or about 75% of the diameter or dimension of the nanosensor, less than or about 50% of the diameter or dimension of the nanosensor, less than or about 25% of the diameter or dimension of the nanosensor, or less than or about 10% of the diameter or dimension of the nanosensor. Additional non-limiting examples of pore sizes can include a diameter or other dimension that is about 99% to about 90% of the diameter or dimension of the nanosensor, about 90% to about 75% of the diameter or dimension of the nanosensor, about 75% to about 50% of the diameter or dimension of the nanosensor, about 50% to about 25% of the diameter or dimension of the nanosensor, about 25% to about 10% of the diameter or dimension of the nanosensor, or about 10% to about 1% of the diameter or dimension of the nanosensor. However, the pores may be even smaller.

FIG. 2B shows a schematic representation of the size exclusion selection of substances passing through the pores 16 and into the chamber 22. As shown, substance 26 has a dimension that is too large to enter the pore 16 and its path 26a does not enter into the pores 16. Substance 28 has a size smaller than the pore 16 and is capable of entering into the chamber 22 as shown by the path 28a; however, the nanosensors 24 do not recognize the substance 28 and no signal is provided. The target substance 30 has a size that allows for entry through the pore 16 and into the chamber 22 as shown by path 30a so as to be capable of interacting with the nanosensors 24. FIG. 2C shows that when the target substance 30 interacts with the nanosensor 24, a signal 32 is provided.

In one embodiment, the nanosensor 34 can be coupled to the polymeric body 12, as shown in FIG. 2B. The nanosensor 34 can be conjugated, associated, linked, or otherwise connected to the container 12. Examples of conjugating the nanosensor 34 to the container can be covalently, ionically, hydrophobic association, hydrophilic association, and the like. The nanosensor 34 can be directly coupled to the container 12 so that the nanosensor 34 contacts the polymeric body. Alternatively, the nanosensor 34 can be coupled to the container 12 through a linker (not shown).

Additionally, the container can be configured as a vessel with at least one internal chamber that retains the nanosensor. The internal chamber of the vessel can be in fluid communication with any external environment in which the vessel resides or is placed in. Such fluid communication between the internal chamber and the external environment allows for fluidic components of the external environment to passively enter into the internal chamber so that an interaction with the nanosensor is possible.

The container and polymeric body can be prepared from a biocompatible polymer. In one instance, the biocompatible polymer can be a biostable polymer. In another instance, the biocompatible polymer can have a degree of biodegradability. Non-limiting examples of biocompatible polymers that can be used in the container and polymeric body can include nylons, poly(alpha-hydroxy esters), polylactic acids, polylactides, poly-L-lactide, poly-DL-lactide, poly-L-lactide-co-DL-lactide, polyglycolic acids, polyglycolide, polylactic-co-glycolic acids, polyglycolide-co-lactide, polyglycolide-co-DL-lactide, polyglycolide-co-L-lactide, polyanhydrides, polyanhydride-co-imides, polyesters, polyorthoesters, polycaprolactones, polyesters, polyanydrides, polyphosphazenes, polyester amides, polyester urethanes, polycarbonates, polytrimethylene carbonates, polyglycolide-co-trimethylene carbonates, poly(PBA-carbonates), polyfumarates, polypropylene fumarate, poly(p-dioxanone), polyhydroxyalkanoates, polyamino acids, poly-L-tyrosines, poly(beta-hydroxybutyrate), polyhydroxybutyrate-hydroxyvaleric acids, copolymers thereof, derivative polymers thereof, monomers thereof, combinations thereof, or the like. Other biocompatible, biodegradable, and/or biostable polymers can be used with or in place of any of the above-referenced polymers. The container can also be water stable so that the container body does not degrade in the presence of water or other aqueous solution. Also, the container can be prepared from polymers that have stability in organic solutions so that the container does not degrade when the medium being tested for the presence of the target substance includes, for example without limitation, an organic solution, organic components, or hydrophobic components.

The polymeric container can be configured to have various shapes and sizes over a broad range. With regard to size, the polymeric container can have a dimension, such as diameter, width, length, height, or the like, that ranges from about 10 nm to about 1 mm. In another option, the dimension can range from about 50 nm to about 100 um. In yet another option, the dimension can range from about 75 nm to about 10 um. In still yet another option, the dimension can range from about 100 nm to about 1 um. Also, larger containers can range between the foregoing values in the micrometer (um) range, millimeter (mm) range, and centimeter (cm range). In some instances certain applications can utilize a container that is larger, equal to, or smaller than any of the recited dimensions.

The polymeric container can be configured to be suspended in a fluid, such as water, sample, biological fluid, or the like. The polymeric container can be suspended in a fluid by attachment of fluid suspending members to the polymeric container. When the medium being tested for the presence of the target substance is water or another aqueous medium, water solubilizing agents (e.g., agents soluble in water) can be associated with the container to provide suspension. Water solubilizing agents can be associated with the container to provide the container with water solubility or water suspendabilty without degrading the container. Non-limiting examples of suspending members or solubilizing agents include highly water soluble polymers, polyetheleneglycols, surfactants, or other water soluble agents being coupled with the container, such as being coupled to the exterior surface.

In one embodiment, a sensor device can include a polymeric container having at least one body wall with an external surface and an internal surface defining an internal chamber. The body wall can include a plurality of pores extending from the external surface to the internal surface such that the internal chamber is in fluid communication with an external environment. At least a portion of the pores can have a first dimension that provides size exclusion selection of substances entering into the chamber. The nanosensors are retained within the internal chamber of the polymeric container. Each nanosensor can have a second dimension that is larger than the first dimension of the pores such that each nanosensor is retained within the internal chamber. At least a first portion of nanosensors can be configured to interact with a target substance so as to provide a signal that can be detected. Optionally, a second portion of nanosensors can be configured to interact with a second target substance so as to provide a second signal that can be detected, where the second signal is distinguishable from the first signal. Any number of different types of nanosensors can be included for detecting any number of different target substances. Barcode quantum materials can be used, for example, for multiplexing when different types of target substances are to be detected.

A sensor device can include a polymeric container having at least one body wall having at least a first surface. The body wall can have a plurality of pores extending from the first surface into the body wall. As such, the pores do not necessarily open into a primary internal chamber; rather, the pores can provide multiple internal chambers within the container. A portion of the pores can have a first dimension that can contain a plurality of nanosensors disposed therein. Each nanosensor can have a second dimension that is the same size, smaller, or larger than the first dimension or the pores so long as the nanosensors are retained within the container and can interact with the target substance to provide the signal.

In one embodiment, the container can also include a barcode quantum material 36 configured for multiplexing as shown in FIG. 2B. The barcode quantum material 36 can be provided within the container 10 in any manner as discussed in connection with the nanosensors 24. Also, the barcode quantum material 36 can be associated with a nanosensor 24. The barcode quantum material 36 can include at least one quantum dot or a plurality and arrangement of quantum dots. The barcode quantum material 36 can provide for multiplexing in that multiple target substances can be detected with multiple nanosensors. The quantum dots can be an assortment of, for example without limitation, cadmium selenide dots, zinc sulfide quantum dots, or the like. Semiconductor nanocrystals, also known as quantum dots, can be used to encode microspheres that subsequently can be used for multiplexed detection of multiple target substances. By combining mixtures of quantum dots with distinct emission wavelengths and intensities, unique spectral ‘barcodes’ are created that enable the high levels of multiplexing required for complex detections, such as specific polynucleotide detection.

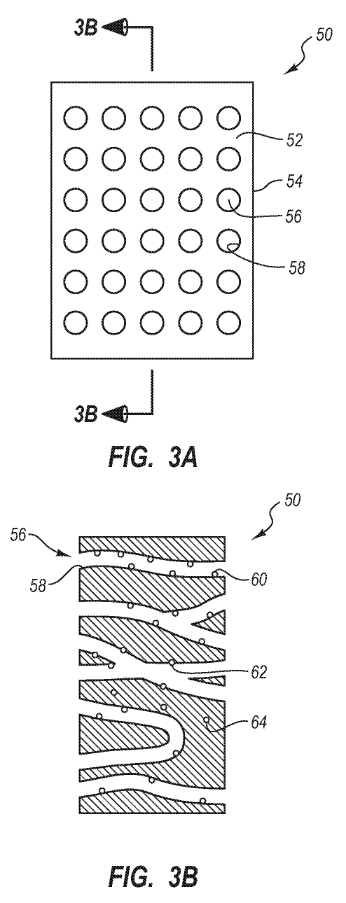

FIGS. 3A-3B show a container 50 defined by a polymeric body 52 with an external surface 54. FIG. 3A, a side view, shows that the polymeric body 52 includes a plurality of pores 56, each being defined by a pore wall 58. FIG. 3B, a cutaway side view of FIG. 3A, shows that the container 50 is substantially devoid of a single, primary chamber. Instead, the plurality of pores 56 cooperate to provide chambers for retention of the nanosensors 60 included therein. As shown, a nanosensor 60 can be coupled to the pore wall 58 of a pore 56. Also, a nanosensor 62 can be at least partially embedded in the pore wall 58 so that a portion protrudes into the pore 56, which allows for interaction with the target substance. Additionally, a nanosensor 64 can be embedded within the pore wall 58 so as to be between pores 56, and the pore wall 58 can be a matrix with interstitial spaces that allow passage of the target substance so as to be capable of interacting with the nanosensor 64 in order to provide the signal. While not shown, a nanosensor can be larger than the pore so as to be retained within a pore 56 and can be in contact with the pore wall 58, and still be capable of interacting with the target substance. The nanosensors described herein that have the same or different element numbers can be the same or different types of nanosensors, and different numbers are used for clarity.

The nanosensors have a high degree of specificity for the target substance. This can include the nanosensor being specific for the target substance so that the signal is provided only when the nanosensor interacts with the target substance, which is an example of strict specificity. Also, less stringent specificity can be used where the nanosensor provides the signal when it interacts with the target substance or a close derivative, analog, salt, or other minor change. Loose specificity can be used when the nanosensor provides a signal when interacting with one of a member of a class or a species of a genus of types of target substances. Additionally, the specificity can be achieved by modulating the percentage of purine nucleotides and pyrimidine nucleotides to determine a suitable temperature to obtain single stranded or double stranded polynucleotides.

Nanosensors are available that provide a signal that is selected from the group consisting of an electronic signal, optical signal, magnetic signal, electrochemical signal, and combinations thereof. Accordingly, a nanosensor can be selected based on the type of signal provided. In different instances, any of the above-referenced signal types can be favorable. The selection of the nanosensor may result in a specific type of signal in instances where the nanosensor interacts with a target substance to provide a specific signal type.

The nanosensor can provide a signal having a first characteristic in the absence of the target substance and then change the signal to a second characteristic upon interaction with the target substance. This can include a first wavelength or first wavelength pattern that is changed to a second wavelength or second wavelength pattern. The nanosensor can have an absorption, transmission, or other emission profile that has a first characteristic, and the characteristic is changed to a second characteristic upon interaction with the target substance. Such a change can be detectable so that the detection of the target substance results from detection in a change in the signal from a first characteristic to a second characteristic.

### II. Polynucleotide Sensor Device

In one embodiment, a sensor device can include a polymeric container having at least one nanosensor configured to sense a polynucleotide retained therein. The polynucleotide nanosensor can be retained within the polymeric container in any manner described herein in connection to any nanosensor. The sensor device can be configured as described herein with the nanosensor having a nucleotide sequence (e.g., probe polynucleotide) that interacts with a target polynucleotide.

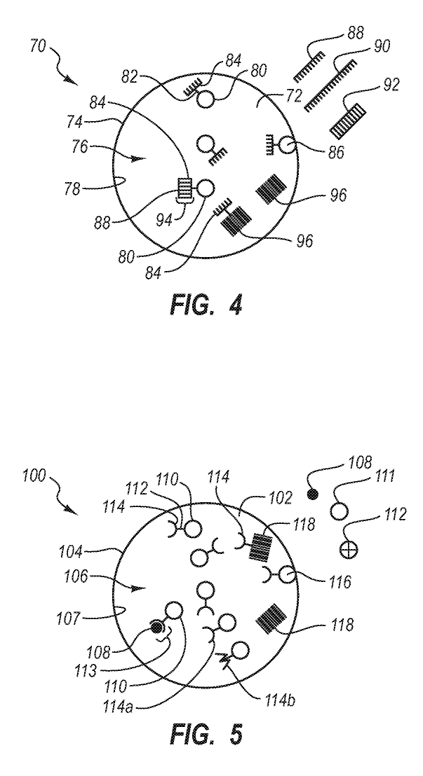

FIG. 4, a cutaway side view, provides a schematic illustration of a sensor device 70 that is configured to interact with and detect a target polynucleotide 88. The sensor device 70 is shown to have a container 72 made from a polymeric material that defines an external surface 74 that is perforated with pores (not shown). The pores can also be holes, conduits, entrances, exits, or the like, and can extend from an external environment into an internal chamber 76 that is defined by at least one internal surface 78. It should be noted that the pores of the sensor device 70 can be configured as any of the pores or conduits as described herein and can function for size exclusion selection of polypeptides entering into the container.

As shown, a long polynucleotide 90 can have a dimension that is too large to traverse the pores and enter the chamber 76. A double stranded polynucleotide 92 may also be too large to enter the chamber 76. On the other hand, the double stranded polynucleotide 92 may enter into the chamber 76, but is inhibited from interacting with the nanosensor 80 due to the duplex configuration. The target polynucleotide 88 has a size that allows for entry through the pores and into the chamber 76. Additionally, the conditions of the medium, such as temperature, tonicity, and the like, can be modulated so that double stranded polynucleotides can unravel into single stranded polynucleotides so as to be capable of interacting with the nanosensor 80. Selective unraveling can be obtained by using the percentage of purine nucleotides and pyrimidine nucleotides to determine a suitable temperature to obtain single stranded polynucleotides.

The chamber 76 is dimensioned so as to include at least one nanosensor 80 disposed and retained therein, and shown to include a plurality of nanosensors 80. The nanosensors 80 can include a probe polynucleotide 84 that has a sequence configured to hybridize, associate, or otherwise interact with the target polynucleotide 88. Optionally, the nanosensor 80 can include a linker 82 that attaches the probe polynucleotide 84 to the nanosensor. The linker can be any type of linking molecule, non-limiting examples of which include polynucleotides, polypeptides, hydrocarbon chains (e.g., C2-C20, C4-C15, C6-C10, or other carbon chain) polysaccharides (e.g., chitan, chitosan, starches, celluloses, carboxy celluloses, etc.), and the like. The length of the linker can be varied as needed depending on the size and configuration of the nanosensor. As shown, the hybridization 94 of the probe polynucleotide 84 of the nanosensor 80 with the target polynucleotide 88 can provide a detectable signal so as to identify that the target polynucleotide 88 is present.

In one embodiment, the nanosensor 86 can be coupled to the container 72. The nanosensor 86 can be conjugated, associated, linked, or otherwise connected to the container 72. Examples of conjugating the nanosensor 86 to the container can be covalently, ionically, hydrophobic association, hydrophilic association, and the like. The nanosensor 86 can be coupled to the container 72 by any type of linking molecule, non-limiting examples of which include polynucleotides, polypeptides, hydrocarbon chains, polysaccharides, and the like.

In one embodiment, the container 72 can also include a barcode quantum material 96 configured for multiplexing. The barcode quantum material 96 can be provided within the container 72 in any manner as discussed in connection with the nanosensors 80. Also, the barcode quantum material 96 can be associated with a nanosensor 80. The barcode quantum material 96 can provide for multiplexing in that multiple target polynucleotides 88 can be detected with multiple nanosensors 80. Also, the barcode quantum material 96 can include a probe polynucleotide 84 coupled to a barcode quantum material 96. The barcode quantum material 96 can be configured and used as described herein.

A target polynucleotide sensor device can include at least one body wall having an external surface and an internal surface defining an internal chamber. The body wall can have a plurality of pores extending from the external surface to the internal surface such that the internal chamber is in communication with an external environment. The internal chamber is dimensioned to contain and retain a plurality of nanosensors that each have a probe polynucleotide that is configured for hybridizing or otherwise associating with a target polynucleotide. The interaction between the probe polynucleotide and the target polynucleotide can provide a signal that can be detected. The nanosensors are dimensioned larger than the pores so that the nanosensors are retained within the internal chamber.

In one embodiment, a polynucleotide sensor device can include a polymeric container having at least one body wall, but being devoid of a primary internal chamber. As such, the polymeric container is configured with a plurality of pores extending from the first surface into the at least one body wall such that a nanosensor as described herein can be provided and retained within the pores or within the body wall. The nanosensors can each have a probe polynucleotide configured for hybridizing or otherwise interacting with a target polynucleotide so as to provide a signal that can be detected. The nanosensors can be dimensioned so as to be retained within the body wall, such as within the pores or within the polymer matrix of the wall.

Similar to other embodiments, a sensor device having a probe polynucleotide for interacting with a target polynucleotide can be characterized by any of the following: at least a portion of the nanosensors are coupled to at least one body wall of the polymeric container; the pores are larger than the target polynucleotide and smaller than the nanosensors; the nanosensors have a high degree of specificity for the target polynucleotide, the high degree of specificity being characterized by at least about 75%, at least about 90%, or at least about 99% complementarity of the target polynucleotide with the probe polynucleotide, or about 50% to about 75%, about 75% to about 90%, or 90% to about 99% complementarity; the interaction between the target polynucleotide and probe polynucleotide provides a signal that is selected from the group consisting of an electronic signal, optical signal, magnetic signal, electrochemical signal, and combinations thereof; the interaction between the target polynucleotide and probe polynucleotide of the nanosensor induces a detectable change in the signal; the container is made from a biocompatible polymer that is biodegradable or biostable polymer; the polymeric container is configured to be suspended in a fluid; the polymeric container is stable in an aqueous solution; the polymeric container is stable in an organic solution; or at least a portion of the nanosensors are imbedded in the body wall.

As used herein, the terms “complementary” and “complementarity” are meant to refer to the ability of polynucleotides to form base pairs with one another. Base pairs are typically formed by hydrogen bonds between nucleotide units in anti-parallel polynucleotide strands. Complementary polynucleotide strands can base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine.

Perfect complementarity or 100% complementarity refers to the situation in which each nucleotide unit of one polynucleotide strand can hydrogen bond with a nucleotide unit of an anti-parallel polynucleotide strand. Less than perfect complementarity refers to the situation in which some, but not all, nucleotide units of two strands can hydrogen bond with each other. For example, for two 20-mers, if only two base pairs on each strand can hydrogen bond with each other, the polynucleotide strands exhibit 10% complementarity. In the same example, if 18 base pairs on each strand can hydrogen bond with each other, the polynucleotide strands exhibit 90% complementarity. “Substantial complementarity” refers to polynucleotide strands exhibiting 79% or greater complementarity, that are selected so as to be non-complementary.

### III. Polypeptide Sensor Device

In one embodiment, a sensor device can include a polymeric container having at least one polypeptide nanosensor (e.g., configured to sense a polypeptide) disposed therein. The polypeptide nanosensor can be retained within the polymeric container in any manner described herein in connection to any nanosensor. The sensor device can be configured as described herein with the nanosensor having a target recognition moiety (e.g., protein, receptor, antibody, antibody fragment, etc.) that interacts with a target polypeptide.

FIG. 5, a cutaway side view, provides a schematic illustration of a sensor device 100 that is configured to interact with and detect a target polypeptide 108. The sensor device 100 is shown to have a container 102 made from a polymeric material that defines an external surface 104 that is perforated with pores (not shown). The pores can also be holes, conduits, entrances, exits, or the like, and can extend from an external environment into an internal chamber 106 that is defined by at least one internal surface 107. It should be noted that the pores of the sensor device 100 can be configured as any of the pores or conduits as described herein and can function for size exclusion selection of polypeptides entering into the container.

As shown, a large polypeptide 111 can have a dimension that is too large to traverse the pores and enter the chamber 106. The target polypeptide 108 has a size that allows for entry through the pores and into the chamber 106. Also, another polypeptide 112 can have a size the same or smaller than the target polypeptide 108 so as to enter into the chamber 106; however, the polypeptide 112 does not have an amino acid sequence that is detected by the nanosensor 110.

The chamber 106 is dimensioned so as to include at least one nanosensor 110 retained therein, and shown to include a plurality of nanosensors 110. The nanosensors 110 can include a target recognition moiety 114 that is configured to bind, associate, or otherwise interact with the target polypeptide 108. Optionally, the nanosensor 100 can include a linker 112 that attaches the target recognition moiety 114 to the nanosensor 110. The linker can be any type of linking molecule, non-limiting examples of which include polynucleotides, polypeptides, hydrocarbon chains (e.g., C2-C20, C4-C15, C6-C10, or other carbon chain) polysaccharides (e.g., chitan, chitosan, starches, celluloses, carboxy celluloses, etc.), and the like. The length of the linker can be varied as needed depending on the size and configuration of the nanosensor. As shown, the interaction 113 of the target recognition moiety 114 of the nanosensor 110 with the target polypeptide 108 can provide a detectable signal so as to identify that the target polypeptide 108 is present. The target recognition moiety 114 can be configured with a first configuration 114a or another configuration 114b. That is, multiple configurations, such as 114a and/or 114b, can be used to interact with the target polypeptide 108.

In one embodiment, the nanosensor 116 can be coupled to the container 102. The nanosensor 116 can be conjugated, associated, linked, or otherwise connected to the container 102 as described herein, such as being coupled to the internal wall 107.

In one embodiment, the container can also include a barcode quantum material 118 configured for multiplexing as described herein. The barcode quantum material 118 can be provided within the container 102 in any manner as discussed in connection with the nanosensors 110. Also, the barcode quantum material 118 can be associated with a nanosensor 110. The barcode quantum material 118 can provide for multiplexing in that multiple target polypeptides 108 can be detected with multiple nanosensors 110. Also, a probe polynucleotide barcode quantum material 118 can include a target recognition moiety 114 coupled to a barcode quantum material 118.

In one embodiment, a polypeptide sensor device can include a polymeric container having at least one body wall with an external surface and an internal surface defining an internal chamber. The body wall can have a plurality of pores extending from the external surface to the internal surface such that the internal chamber is in communication with an external environment. A plurality of nanosensors can be retained in the internal chamber with each nanosensor having a target recognition moiety configured for interacting with a target polypeptide. The interaction between the target recognition moiety and the target polypeptide can provide a signal that can be detected. Each nanosensor can have a dimension that is larger than the dimensions of the pores such that each nanosensor is retained within the internal chamber.

In one embodiment, a polypeptide sensor device can include a polymeric container having at least one body wall with at least a first surface, but being devoid of a primary internal chamber. As such, the polymeric container is configured with a plurality of pores extending from the first surface into the body wall such that a nanosensor as described herein can be retained within the pores or within the body wall. The nanosensors can each have a target recognition moiety configured for interacting or otherwise associating with a target polypeptide so as to provide a signal that can be detected. The nanosensors can be dimensioned so as to be retained within the body wall, such as within the pores or within the polymer matrix of the wall.

Similar to other embodiments, nanosensors having a target recognition moiety for interacting with a target polypeptide can be characterized by any of the following: at least a portion of the nanosensors are coupled to a body wall of the polymeric container; the pores are larger than the target polypeptide and smaller than the nanosensors; the nanosensors have a high degree of specificity for the target polypeptide; the signal that is selected from the group consisting of an electronic signal, optical signal, magnetic signal, electrochemical signal, and combinations thereof; the interaction between the target recognition moiety and the target polypeptide induces a detectable change in the signal; the body wall includes a biocompatible and biostable polymer; the polymeric container is configured to be suspended in a fluid; the polymeric container is stable in an aqueous solution; the polymeric container is stable in an organic solution; or at least a portion of the nanosensors of the plurality are imbedded in the at least one body wall.

### IV. Cell Sensor Device

In one embodiment, a cell sensor device can include a polymeric container having at least one cell nanosensor (e.g., configured to sense a cell) retained therein. The cell nanosensor can be retained within the polymeric container in any manner described herein in connection to any nanosensor. The sensor device can be configured as described herein with the nanosensor having a cell recognition moiety (e.g., protein, receptor, antibody, antibody fragment, ligand, etc.) that interacts with a target cell. The cell to be detected can be any type of cell from any organism.

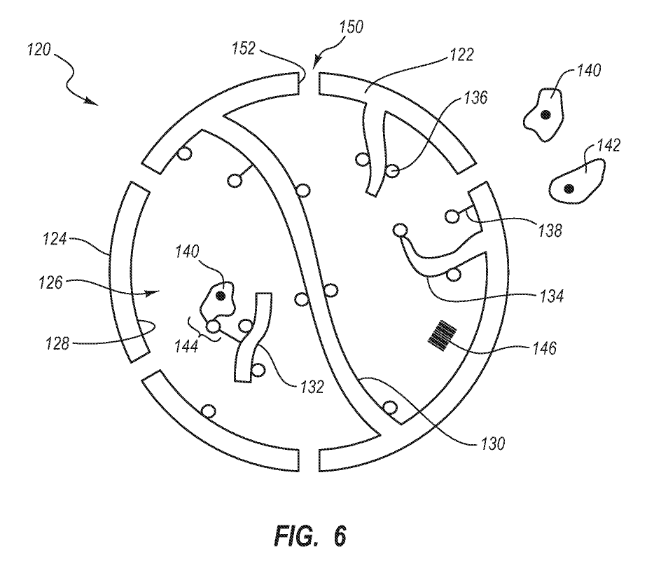

FIG. 6 provides a schematic illustration of a sensor device 120 that is configured to interact with and detect a target cell 140. The sensor device 120 is shown to have a container 122 made from a polymeric material that defines an external surface 124 that is perforated with pores 150 that are defined by pore walls 152. The pores 150 can also be holes, conduits, entrances, exits, or the like, and can extend from an external environment into an internal chamber 126 that is defined by at least one internal surface 128. The pores 150 can be large enough to allow for cells, living or dead, to enter into the container 122. It should be noted that the pores 150 of the sensor device 120 can be configured as any of the pores or conduits as described herein and can function for size exclusion selection of cells entering into the container. The container 122 shown in FIG. 6 can include interior body walls 130, floating walls 132, and walls 134 that are coupled to the internal surface 128. Any of these walls can be attached to the container or free floating within the container so as to be retained therein.

While not shown, a non-target cell can have a dimension that is too large to traverse the pores 150 and enter the chamber 126. Such a large non-target cell is prevented from entering into the chamber 26 by size exclusion. The target cell 140 has a size that allows for entry through the pores 150 and into the chamber 126. Also, another cell 142 can have a size the same or smaller than the target cell 140 so as to enter into the chamber 126; however, the cell 142 does not have a cell surface component or other feature that is detected by the sensor device 120.

The chamber 126 is dimensioned so as to include at least one nanosensor 136 disposed and retained therein, and shown to include a plurality of nanosensors 136. The nanosensors 136 can include a target recognition moiety (not shown) that is configured to bind, hybridize, associate, or otherwise interact with the target cell 140. Optionally, the nanosensor 136 can include a linker 138 that attaches the nanosensor 136 to the container 122. As shown, the interaction 144 of the target cell 140 with the nanosensor 136 can provide a detectable signal so as to identify that the target cell 140 is present.

The container 120, chamber 126, and/or pores 150 are dimensioned so as to include at least one cell therein, and may include a number of cells. For example, and without limitation, the container 120, chamber 126, and/or pores 150 are dimensioned to include at least about 10 cells, at least about 100 cells, at least about 1,000 cells, at least about 10,000 cells, at least about 100,000 cells, or at least about 1 million or more cells.

In one embodiment, the container can also include a barcode quantum material 146 configured for multiplexing. The barcode quantum material 146 can be retained within the container 122 in any manner as discussed in connection with the nanosensors 136. Also, the barcode quantum material 146 can be associated with a nanosensor 136. The barcode quantum material 146 can provide for multiplexing in that multiple target cells 140 can be detected with multiple nanosensors 136. The barcode quantum material 146 can also include all the features of a nanosensor 136 and function similarly.

In one embodiment, a cell sensor device can include a biocompatible polymeric macroparticle having at least one body wall defining a plurality of cell passageways extending into the polymeric macroparticle. The cell passageways (e.g., pores) having a shape and size sufficient for passing a cell therethrough. A plurality of nanosensors can be retained in the plurality of cell passageways and coupled to the body wall of the polymeric macroparticle such that the nanosensor is retained within the polymeric macroparticle. Additionally, the nanosensors can be coupled to any portion of the macroparticle or can be embedded or partially embedded within a body wall of the macroparticle. The nanosensors are configured to interact with a target cell so as to provide a signal that can be detected.

The cell sensor device can be characterized by any of the following: the nanosensors include a target recognition moiety that interacts with a surface component of the target cell; the target recognition moiety interacts with a protein present on the surface of the target cell; the target recognition moiety interacts with a receptor present on the surface of the target cell; the nanosensor has a high degree of specificity for a receptor on the cell; the interaction of the target cell and nanosensor provides a signal selected from the group consisting of an electronic signal, optical signal, magnetic signal, electrochemical signal, and combinations thereof; the interaction between the target cell and a nanosensor of the plurality of nanosensors induces a detectable change in the signal; the body wall includes a biostable polymer; the polymeric macroparticle has a size sufficient such that at least about 10 cells can simultaneously enter into the cell passageways; the polymeric macroparticle has a size sufficient such that at least about 100 cells, at least about 1,000 cells or more can simultaneously enter into the cell passageways; the polymeric macroparticle is configured to be suspended in a fluid; the polymeric macroparticle is compatible and stable in an in vitro environment; the polymeric container is compatible and stable in an in vivo environment; or a portion of the nanosensors are imbedded in the at least one body wall.

### V. Nanosensors

The nanosensors that can be included in the sensor devices described herein represent a broad class of sensors that can be employed to detect a target substance. The nanosensors can include those described herein as well as those well known in the art and those later developed. The nanosensors described herein can be complexed with a retaining entity that is sized to be retained within the sensor device as described. Additionally, a plurality of the nanosensors can be retained within the sensor device to increase sensitivity to detect the target at lower concentrations.

In one embodiment, a nanosensor can include a nanowire. Such nanowires have high surface-to-volume ratios, and can be synthesized from ceramics and polymers. The nanowires can be used to detect chemical agents (e.g., pesticides), microorganisms (e.g., E. coli, Giardia), and mineral compounds. The nanowires can include surface or other interfacial chemical modifications to achieve selectivity for a target substance. As such, receptors, ligands, epitopes, antibodies, antibody fragments, and the like can be included on nanowires.

In one embodiment, a nanosensor is a molecule or ion sensor. Such molecular sensors can be configured to detect the presence of specific substances, and combine the properties of supramolecular receptors, as they specifically recognize a specific substance, with the ability to produce a measurable signal. Optical signals based on changes of absorbance, transmission, or fluorescence are the most frequently utilized because of their simple applications and use of common instruments. The molecular sensors can change absorbance, particularly of color, when interacting with a target substance. Such changes can be used to detect the presence of the target substance. The use of molecular sensors that provide or change fluorescence emission provides very high sensitivity of the sensor device. One category of fluorescence chemosensors includes classical fluorescence chemosensors made from molecules in which a supramolecular receptor and a fluorescence dye are part of the same molecule. Another class is that of self-organized fluorescence chemosensors, which are obtained by the spontaneous self-organizing of the sensor components.

A fluorescence chemosensor, ATMCA, can be obtained by coupling an anthrylmethyl group to an amino nitrogen of TMCA (2,4,6-triamino-1,3,5-trimethoxycyclohexane), a tripodal ligand selective for divalent first-row transition metal ions in water. The ATMCA ligand can act as a versatile sensor for Zn and Cu ions, where the sensing ability can be switched by simply tuning the operating conditions. At pH 5, ATMCA detects copper ions in aqueous solutions by the complexation-induced quenching of the anthracene emission. Metal ion concentrations <1 μM can be readily detected and very little interference is exerted by other metal ions. At pH 7, ATMCA signals the presence of Zn ions at concentrations <1 μM by a complexation-induced enhancement of the fluorescence. Such a chemosensor is a nanosensor, and can be used in the sensor devices as described herein. The ATMCA can be complexed with a retaining entity that is sized to be retained within the sensor device as described. Additionally, a plurality of ATMCA can be retained within the sensor device to increase sensitivity to detect the target at lower concentrations.

Additionally, the [Zn(ATMCA)]₂₊ complex can act as a fluorescence nanosensor for specific organic species, such as selected dicarboxylic acids and nucleotides, by the formation of ternary ligand/zinc/substrate complexes. The oxalate anion can be detected in concentrations <0.1 mM. Nucleotides containing an imide or amide function can be detected with the nanosensor, and the nanosensor has high sensitivity for guanine derivatives. Moreover, the ATMCA.Zn(II) complex is an effective and selective sensor for vitamin B13 (orotic acid) in sub-micromolar concentrations. The formation of the complex with vitamin B13 leads to the quenching of the fluorescence emission of anthracenyl residue.

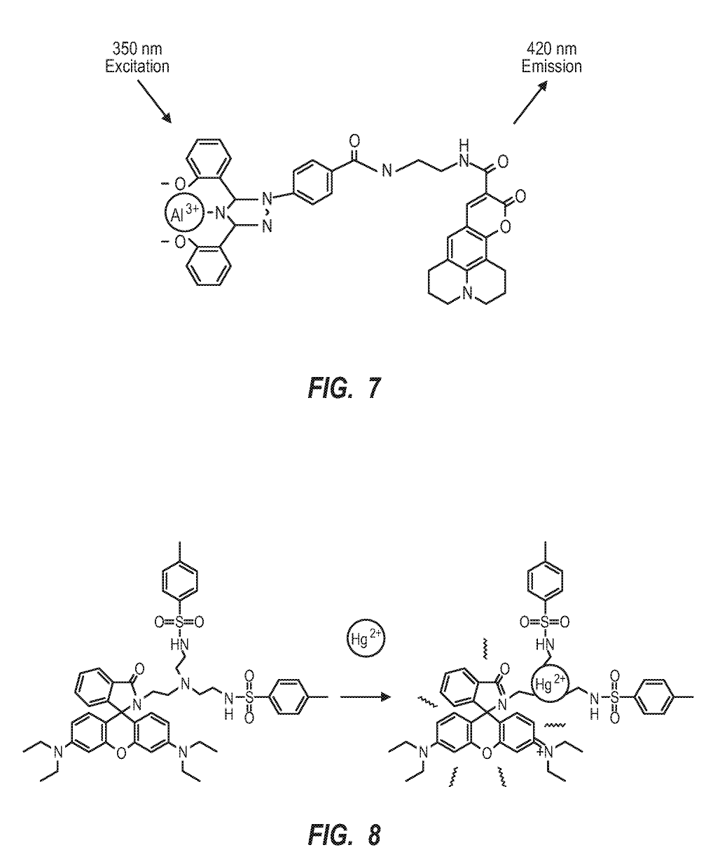

Another non-limiting example of a nanosensor is a Foster resonance energy transfer (FRET) amplified chemosensor as shown in FIG. 7. The sensing activity includes the binding of Al(III) to a 3,5-bis(ortho-hydroxyphenyl)-1,2,4-triazole group, and produces a chelation induced fluorescence enhancement (CHEF). The 3,5-bis(ortho-hydroxyphenyl)-1,2,4-triazole group can be used as a nanosensor as described herein. Also, conjugation of the 3,5-bis(ortho-hydroxyphenyl)-1,2,4-triazole group with coumarine 343 allows the amplification of the fluorescence signal via a FRET process.

Another non-limiting example of a nanosensor is a self-assembled chemosensor for Cu(II) having decylglycylglycine and ANS chromophore in close proximity. The Cu(II) selective receptor (decylglycylglycine) and a chromophore (ANS) can be in close proximity with CTABr surfactant so as to aggregate. Also, the components can be coupled to a microparticle, such as silica. The close proximity produces fluorescence quenching after Cu(II) addition in concentrations below the micromolar range. Commercially available particles (e.g., 20 nm diameter) can be functionalized with triethoxysilane derivatives of selective Cu(II) ligands and fluorophores. The sensor components can be coupled to the particle surface to provide spatial proximity to signal Cu(II) by quenching of the fluorescence emission. In 9:1 DMSO/water solution, the coated silica nanoparticles (CSNs) selectively detect copper ions down to nanomolar concentrations, and the operative range of the sensor can be tuned by the simple modification of the components ratio.

A tren-based tripodal chemosensor bearing a rhodamine and two tosyl groups, as shown in FIG. 8, can be prepared to detect metal ions. Detection can be observed through UV/vis and fluorescence spectroscopies. Addition of a Hg²⁺ ion to the nanosensor can provide a visual color change as well as significantly enhanced fluorescence, while other ions including Pb²⁺, Zn²⁺, Cu²⁺, Ca²⁺, Ba²⁺, Cd²⁺, Co²⁺, Mg²⁺, Ag⁺, Cs⁺, Li⁺, and Na⁺ induced no or much smaller color/spectral changes. As such, the nanosensor is an Hg²⁺-selective fluorescent nanosensor. Such a nanosensor can be used as described herein.

Additionally, quantum dots or barcode quantum materials having specific arrangements and fluorescent augmentations can be used as nanosensors. Zinc sulfide quantum dots, though not quite as fluorescent as cadmium selenide quantum dots, can have augmented fluorescence by including other metals such as manganese and various lanthanide elements. The quantum dots can become more fluorescent when they bond to their target, such as target substances, polynucleotides, polypeptides, and cells. The quantum dots or barcode quantum materials having the quantum dots can be used in ultrasensitive nanosensors. Different high-quality quantum dot nanocrystals (ZnS, CdS, and PbS) can be tagged to a target recognition moiety (e.g., probe polynucleotides, ligands, receptors, antibodies, antibody fragments, etc.) for on-site voltammetric stripping measurements of multiple antigen targets. The quantum dots or barcode quantum materials can have distinct redox potential and yield highly sensitive and selective stripping peaks at −1.11 V (Zn), −0.67 V (Cd) and −0.52 V (Pb) at a mercury-coated glassy carbon electrode compared to references. The change in position and size of these peaks reflect the presence and concentration level of the corresponding target.

A nanosensor can include a nanotube having a target recognition moiety that interacts with a target substance, polynucleotide, polypeptide, or cell. Accordingly, the target recognition moiety is configured for interacting with the target. The nanotube, such as a carbon nanotube, can have a first vibrational energy when the target recognition moiety is not interacting with the target and then have a second vibrational energy when the target recognition moiety interacts with the target. The difference between the first and second vibrational energy is measurable and detection of the difference can provide an indication that the target is present. Thus, any type of target recognition moiety can be applied to a nanotube in order to have a nanosensor that can be used as described herein. Energies other than vibrational energy may also be used for detection purposed.

In one embodiment, a nanosensor can be configured as a “core-satellite” structure, which resembles a planet (gold) with numerous smaller moons (particles) tethered to it by tiny strands of polynucleotides having probe polynucleotide sequences. The probe polynucleotide sequences can be configured for hybridizing with the target polynucleotide so as to have suitable complementarity. Gold core particles and smaller satellite particles of various materials are mixed together in solution with the probe polynucleotides and under controlled circumstances assemble themselves into the desired core-satellite structure. Following assembly, the structures are can be used to detect new strands of polynucleotides of various lengths. The probe polynucleotide tethers between the gold core and particles contract or expand when in the presence of the target polynucleotide. As the particles move in relation to the gold core, the optical properties of the structure change, and thereby provide a signal that can be detected.

In one embodiment, a nanosensor can be a bio-barcode nanosensor. A bio-barcode nanosensor includes a nanosensor that includes a series of barcode oligonucleotides. The barcode oligonucleotides can correspond to a specific target, and interaction of the target with the nanosensors releases one or more of the bio-barcodes, which can be detected.

In one embodiment, a nanosensor can include a nano-gap capacitor. Nan-gap capacitors can be fabricated using silicon nanolithography. A target recognition moiety is immobilized on the nano-gap capacitor in a manner that allows for interaction with the target substance. When the target substance interacts with the target recognition moiety, the capacitance changes in a detectable manner. As such, the nano-gap capacitor is configured to change the detected signal upon interaction of the target substance and a nanosensor.

In one embodiment, a nanosensor can include a nano-cantilever. A target recognition moiety is immobilized on the nano-cantilever in a manner that allows for interaction with the target substance. When the target substance interacts with the target recognition moiety, the deflection properties, vibrational properties, or response to probe signals changes in a detectable manner. Thus, a nano-cantilever can be coupled to a target substance recognition moiety such that interaction of the target substance and the recognition moiety changes the detected signal of the nano-cantilever.

In one embodiment, a sensor system can include any sensor device as described herein that includes a nanosensor in a polymeric container as described herein, and can include a monitor configured to detect a signal that indicates the nanosensor has sensed the target substance. The monitor can be selected based on the type of signal provided by the nanosensor.

### VI. Detecting Target Substances.

In one embodiment, a method of detecting a target substance with a sensor device can be performed with a sensor device as described herein that includes a nanosensor. The sensor device can be placed in a medium to determine whether or not the target substance is present. When the nanosensor of the sensor device interacts with a target substance, a signal is provided. As such, detecting the signal provides an indication that the presence of the target substance in the medium. Optionally, the medium can be selected from the group consisting of water, air, biological sample, hydrocarbon, combinations thereof, and other similar media.

Additionally, the method can further include tagging the target substance with a marker that interacts with the sensor device so as to provide the signal. In various systems, a donor and acceptor can be used as a marker pair, where the target substance is modified to include one of the donor and acceptor and the nanosensor has the other. Close proximity or association of the donor and acceptor provides the detectable signal. For example, a target nucleic acid can be tagged with the marker, which is either the donor or acceptor, and the probe polynucleotide has the other. When the target hybridizes with the probe, the signal is provided.

The method of detecting a target substance can also include determining an amount or concentration of the target substance in the medium. Quantification of the signal or change in signal can be used to determine the amount or concentration of the target substance. Also, the signal can be compared to a control or control set in order to quantify or quantitate the amount or concentration of the target substance.

The method of detecting a target substance can include the use of a probe signal that induces the detection signal to be provided or to change the signal. As such, a probe signal can be directed into the medium to the nanosensor so as to induce at least one nanosensor to provide the signal. The probe signal can provide energy that is changed by the nanosensor in a detectable manner. For example, light of a broad or specific wavelength can be directed into the medium, and the obtained absorbance, transmittance, or fluorescence can be the signal provided as a result of the probe signal.

In some instances, the sensor device or medium may be processed so as to induce at least one nanosensor to provide the signal. Such processing can vary depending on the nanosensor.

In one embodiment, a method of detecting a target polynucleotide with a sensor device can include providing a sensor device that includes a nanosensor having a probe polynucleotide being retained in a polymeric container as described herein; placing the sensor device in a medium; and detecting the signal so as to indicate the presence of the target polynucleotide in the medium. Optionally, the medium can be selected from the group consisting of water, air, biological sample, hydrocarbon, combinations thereof, and other similar media. Additionally, the method can further include tagging the target polynucleotide with a marker that interacts with the sensor device so as to provide the signal. Also, the method can further include determining an amount or concentration of the target polynucleotide in the medium. The method can also include directing a probe signal into the medium so as to induce at least one nanosensor to provide the signal, and/or processing the sensor device so as to induce at least one nanosensor to provide the signal.

In one embodiment, a method of detecting a target polypeptide with a sensor device can include providing a sensor device that includes a nanosensor having a target recognition moiety being retained in a polymeric container as described herein; placing the sensor device in a medium; and detecting the signal so as to indicate the presence of the target polypeptide in the medium. Optionally, the medium can be selected from the group consisting of water, air, biological sample, hydrocarbon, combinations thereof, and other similar media. Additionally, the method can further include tagging the target polypeptide with a marker that interacts with the sensor device so as to provide the signal. Also, the method can further include determining an amount or concentration of the target polypeptide in the medium. The method can also include directing a probe signal into the medium so as to induce at least one nanosensor to provide the signal, and/or processing the sensor device so as to induce at least one nanosensor to provide the signal. In one embodiment, a method of detecting a target cell with a sensor device can include: providing a sensor device as described herein; placing the sensor device in a medium; and detecting the signal so as to indicate the presence of the target cell. The medium can be selected from the group consisting of a cell culture medium, a serum-free cell culture medium, a cell culture washing fluid, a biological sample, a living animal, a dead animal, and combinations thereof. Also, the target cell can be tagged with a marker that interacts with the sensor device so as to provide the signal. Furthermore, the method can also include: determining an amount or concentration of the target cell in the medium; directing a probe signal into the medium so as to induce at least one nanosensor to provide the signal; or processing the sensor device so as to induce at least one nanosensor to provide the signal.

### VII. Manufacturing Sensor Devices

The sensor devices as described herein can be prepared by various methods of encapsulating or including a particle in a container so as to be retained within the container. The container can include a polymer, which can be porous in some instance. In other instances, the polymer can be substantially devoid of pores.

In one embodiment, a method of making a sensor device can include providing a plurality of nanosensors, and forming a polymeric container around the plurality of nanosensors. The polymeric container can be configured to include at least one body wall having an external surface and an internal surface defining an internal chamber. The body wall can be prepared to have a plurality of pores extending from the external surface to the internal surface such that the internal chamber is in communication with an external environment. Also, the pores can be sized so that each nanosensor is retained within the internal chamber.

General manufacturing methods can be used to prepare the sensor device. One general manufacturing method can include extruding a composition having the nanosensors and a polymeric composition so as to produce the sensor device. Another method includes spraying a polymeric composition onto the nanosensors to form the container around the nanosensors. Yet another method includes aggregating the nanosensors together with a removable aggregator, dipping the aggregated nanosensors into a polymeric composition, and removing the aggregator so as to disaggregate the nanosensors. Also, suitable polymer solutions having the nanosensors can be lyophilized to obtain the porous container. Alternatively, two lyophilized polymer compositions can have the nanosensors included thereon or therein and then the two compositions are sandwiched together. Additionally, the sensor device can be obtained by inkjet printing a polymeric composition onto the nanosensors. Alternatively, the nanosensor composition can be inkjet printing onto a polymeric sheet, and the sheet can be formed into a container with itself by folding or be coupled to another polymeric sheet to form a pouch. Moreover, a polymeric composition can be deposited onto and around the nanosensors. Furthermore, a method of polymerizing a polymeric precursor in the presence of the nanosensors can obtain the sensor device.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope. All references recited herein are incorporated herein in their entirety by specific reference.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.”

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A sensor device comprising:
a porous polymeric container; and
at least one nanosensor included and retained within the porous polymeric container.

2. A sensor device as in claim 1, wherein the nanosensor is configured to interact with a target substance so as to provide a signal that can be detected.

3. A sensor device as in claim 2, wherein the target substance is one of an organic molecule, inorganic molecule, atom, ion, nucleotide, polynucleotide, amino acid, polypeptide, protein, receptor, antibody, antibody fragment, cell, cell surface component, ligand, or combinations thereof.

4. A sensor device as in claim 3, wherein the target substance is a target polynucleotide and the nanosensor includes a probe polynucleotide configured to hybridize with the target polynucleotide.

5. A sensor device as in claim 3, wherein the target substance is a target polypeptide and the nanosensor includes a target recognition moiety configured to interact with the target polypeptide.

6. A sensor device as in claim 3, wherein the target substance is a target cell, and the nanosensor includes a target recognition moiety configured to interact with a cell surface component of the target cell.

7. A sensor device as in claim 6, wherein the cell surface component is one of a protein, epitope, receptor, cell membrane component, lipid, or combinations thereof.

8. A sensor device as in claim 1, wherein the nanosensor is retained within an internal chamber of the porous polymeric container.

9. A sensor device as in claim 1, wherein the nanosensor is retained within a pore of the porous polymeric container.

10. A sensor device as in claim 1, wherein the nanosensor is coupled to the porous polymeric container.

11. A sensor device as in claim 1, wherein pores of the porous polymeric container fluidly couple an external environment with an internal chamber of the polymeric container.

12. A sensor device as in claim 1, wherein pores of the porous polymeric container are dimensioned to be larger than a target substance and smaller than a nanosensor such that the target substance can pass through the pores so as to interact with the nanosensor.

13. A sensor device as in claim 5, further comprising a barcode quantum material configured for multiplexing, said barcode quantum material being included within the porous polymeric container.

14. A sensor device as in claim 2, wherein the nanosensor includes a barcode quantum material that is configured to interact with the target substance.

15. A sensor as in claim 2, wherein the signal is selected from the group consisting of an electronic signal, optical signal, magnetic signal, electrochemical signal, and combinations thereof.

16. A sensor device as in claim 2, wherein the interaction between the target substance and a nanosensor induces a detectable change in the signal.

17. A sensor device as in claim 1, wherein porous polymeric container includes a biocompatible and biostable polymer.

18. A sensor device as in claim 1, wherein the nanosensor is characterized by having at least one of the following:
an oligonucleotide probe configured to interact with the target substance being a nucleotide sequence;
a target substance recognition moiety;
a polypeptide receptor configured to interact with the target substance;
a bio-barcode configured to release barcode oligonucleotides upon interaction of a nanosensor of the plurality with the target substance;
a quantum dot;
a barcode quantum material;
a nanotube coupled to a target substance recognition moiety such that interaction of the target substance and the recognition moiety changes the detected signal of the nanotube;
a nano-gap capacitor configured to change the detected signal upon interaction of the target substance and a nanosensor of the plurality; or
a nano-cantilever coupled to a target substance recognition moiety such that interaction of the target substance and the recognition moiety changes the detected signal of the nano-cantilever.

19. A sensor device comprising:
a porous polymeric container; and
at least one nanosensor included and retained within the porous polymeric container, said nanosensor including a probe polynucleotide configured to hybridize with a target polynucleotide.

20. A sensor device as in claim 19, wherein the probe polynucleotide of the nanosensor has a high degree of specificity for the target polynucleotide, the high degree of specificity being characterized by at least 90% complementarity.

21. A sensor device comprising:
a porous polymeric container; and
at least one nanosensor included and retained within the porous polymeric container, said nanosensor including a target recognition moiety configured to interact with a target polypeptide.

22. A sensor device as in claim 21, wherein the target recognition moiety is one of a polypeptide, protein, receptor, antibody, antibody fragment, ligand, or combinations thereof.

23. A sensor device comprising:
a porous polymeric container; and
at least one nanosensor included and retained within the porous polymeric container, said nanosensor including a target recognition moiety configured to interact with a cell surface component of a target cell.

24. A sensor device as in claim 23, wherein the cell surface component is one of a protein, epitope, receptor, cell membrane component, lipid, or combinations thereof.

25. A sensor system comprising:
the sensor device as in claim 1; and
a monitor configured to detect a signal emitted by the sensor device.

26. A method of detecting a target substance with a sensor device, the method comprising:
providing a sensor device as in claim 1;
placing the sensor device in a medium; and
detecting a signal provided by a target substance interacting with the nanosensor of the sensor device so as to indicate the presence of the target substance in the medium.

27. A method as in claim 26, wherein the medium is selected from the group consisting of water, air, biological sample, hydrocarbon, and combinations thereof.

28. A method as in claim 26, further comprising tagging the target substance with a marker that interacts with the nanosensor of the sensor device so as to provide the signal.

29. A method as in claim 26, further comprising determining an amount or concentration of the target substance in the medium.

30. A method as in claim 26, further comprising directing a probe signal into the medium so as to induce at least one of the nanosensors to provide the signal.

31. A method of making a sensor device, the method comprising:
providing a nanosensor;
providing a porous polymeric container; and
combining the nanosensor and the porous polymeric container such that the nanosensor is retained within the porous polymeric container.

32. A method as in claim 31, further comprising configuring the nanosensor to interact with a target substance so as to provide a signal that can be detected.

33. A method as in claim 32, wherein the target substance is one of an organic molecule, inorganic molecule, atom, ion, nucleotide, polynucleotide, amino acid, polypeptide, protein, receptor, antibody, antibody fragment, cell, cell surface component, ligand, or combinations thereof.

34. A method as in claim 33, further comprising configuring the nanosensor to include a probe polynucleotide that hybridizes with a target polynucleotide.

35. A method as in claim 33, further comprising configuring the nanosensor to include a target recognition moiety that interacts with a target polypeptide.

36. A method as in claim 33, further comprising configuring the nanosensor to include a target recognition moiety that interacts with a cell surface component of a target cell.

37. A method as in claim 36, wherein the cell surface component is one of a protein, epitope, receptor, cell membrane component, lipid, or combinations thereof.

38. A method as in claim 31, further comprising preparing the porous polymeric container to have an internal chamber and including the nanosensor in the internal chamber.

39. A method as in claim 31, further comprising configuring pores of the porous polymeric container to have dimensions smaller than dimensions of the nanosensor such that the nanosensor is retained within the porous polymeric container.

40. A method as in claim 31, further comprising coupling the nanosensor to the porous polymeric container.

41. A method as in claim 31, further comprising configuring pores of the porous polymeric container to be larger than a target substance and smaller than the nanosensor such that the target substance can pass through the pores so as to interact with the nanosensor.

42. A method as in claim 31, further comprising including a barcode quantum material configured for multiplexing within the porous polymeric container.

43. A method as in claim 31, further comprising preparing the porous polymeric container from a biocompatible and biostable polymer.

44. A method as in claim 31, wherein the polymeric container and nanosensor are combined by at least one of the following:
mixing the nanosensor into a polymeric material that is prepared into the polymeric container;
extruding a composition having the nanosensors and a polymeric composition;
spraying a polymeric composition onto the nanosensor;
spraying a nanosensor composition onto a polymer composition;
aggregating nanosensors together with a removable aggregator, dipping the aggregated nanosensors into a polymeric composition, and removing the aggregator so as to disaggregate the nanosensors;
lyophilizing a solution having the nanosensors and a polymeric composition;
inkjet printing a polymeric composition onto the nanosensors;
inkjet printing a nanosensor composition onto a polymeric member;
depositing a polymeric composition onto the nanosensors; or
polymerizing a polymeric precursor in the presence of the nanosensors.
